SEQUENCE LISTING (6 sequences)

SEQ ID NO: 1 - DNA, 10 bp, Artificial Sequence
  Description of Artificial Sequence  Synthetic Primer
agaagcttct

SEQ ID NO: 2 - DNA, 10 bp, Artificial Sequence
  modified_base (1)..(10): n = a, c, g or t/u
ngaannttcn

SEQ ID NO: 3 - DNA, 10 bp, Artificial Sequence
  modified_base (1)..(10): n = a, c, g or t/u
nttcnngaan

SEQ ID NO: 4 - DNA, 14 bp, Artificial Sequence
  Description of Artificial Sequence  Synthetic Primer
ctggaatttctaga

SEQ ID NO: 5 - DNA, 14 bp, Artificial Sequence
  modified_base (3)..(14): n = a, c, g or t/u
ctngaanntacnnn

SEQ ID NO: 6 - DNA, 15 bp, Artificial Sequence
  Description of Artificial Sequence  Synthetic Primer
agaacgttctagaac

(19) United States
(12) Patent Application Publication
(10) Pub. No.: US 2009/0105170 A1
(43) Pub. Date: Apr. 23, 2009

(54) Multiple Heat Shock Elements

(75) Inventors: Thomas Czerny, Vienna (AT); Nargessadat Aghaallaei, Vienna (AT); Baubak Bajoghli, Vienna (AT)

Correspondence Address: FULBRIGHT & JAWORSKI L.L.P., 600 CONGRESS AVE., SUITE 2400, AUSTIN, TX, 78701, US

(73) Assignee: VETERINARMEDIZINISCHE UNIVERSITAT WIEN, Vienna (AT)

(21) Appl. No.: 11/568,168

(22) PCT Filed: Apr. 12, 2005
(86) PCT No.: PCT/EP05/51608

§ 371 Date: Oct. 20, 2006

(30) Foreign Application Priority Data
Apr. 20, 2004 (AT) A 674/2004

(51) Int. Cl. A61K 48/00; C07H 21/00; A01H 5/00; C12Q 1/68; A61P 43/00; A01K 67/027; C12P 19/34

(52) U.S. Cl. 514/44; 536/23.1; 800/298; 800/13; 435/91.1; 435/6

(57) ABSTRACT

A DNA molecule is provided which comprises at least 2 consensus sequences, each consensus sequence consisting of 3 pentameric units, said pentameric units having a sequence XGAAY or an inverse sequence Y′TTCX′, X being selected from the group consisting of A, T, G, and C, and Y of at least one, preferably two, still preferred all three, of said 3 pentameric units of at least one consensus sequence being selected from the group consisting of A, T, and C, the Y of the remaining pentameric units of said at least one consensus sequence being selected from the group consisting of A, T, G, and C, whereby in the case that said DNA molecule comprises more than 6 consensus sequences, Y of all pentameric units is selected from the group consisting of A, T, G, and C.

The present application relates to a DNA molecule, the use of a DNA molecule in an expression system and a method for producing an expression system.

Complicated gene regulatory networks are active during embryonic development. The resulting timing of gene activity critically determines gene function. This timing determines both, the presence of an inducing signal, as well as the competence of a tissue to respond to the signal. For example, signal transduction pathways involving Fgf and Wnt family members are known to have numerous functions during embryonic development. Misexpression experiments interfering with these pathways can therefore have quite opposing results depending on the time window of activity. Of major importance for these gain-of-function experiments are therefore effective induction systems, which can be controlled from outside of the embryo.

Inducible misexpression systems consist of two components: An inducible transcription factor and a promoter responsive for this transcription factor. In the cases of hormone-inducible systems, the tet-system, lac promoters and the rapalog-system, one component is affected by an externally added drug and has to be expressed constitutively, whereas the second one containing the inducible promoter together with the gene of interest has to be transcriptionally inactive in the uninduced state. These opposed levels of transcriptional activity for the two components normally prevent a combination within a single DNA construct and requires separate integration into the genome. Successful application in vivo therefore normally depends on two transgenic lines, which have to be crossed. On the contrary, heat shock protein (HSP) promoters are induced by endogenous factors, thereby reducing the system to a single ectopic DNA construct. Thus HSP promoters provide a simple one-component system for inducible misexpression. In particular, systems like fish or insects are ideal for the induction of a heat shock response at elevated temperatures.

Heat shock activation is a highly conserved response to cellular stress. Heat shock proteins, which function as chaperoning, help the cell to survive the stress situation. The activation of this response is regulated at the transcriptional level and heat shock elements (HSE), short sequences present in all HSP promoters have been identified to be essential for stress inducibility. HSEs contain multiple copies of the 5 base pairs sequence NGAAN, detailed mutational analysis identified AGAAC as the optimal sequence (Cunniff and Morgan, The Journal of Biological Chemistry, 268 (11) (1993), 88317-8324). The number of pentameric units in an HSE can vary, but a minimum of 3 is required for efficient heat inducible expression. Positioned upstream of a heterologous promoter, HSEs can confer heat stress inducibility to heterologous promoters. Heat shock factor 1 (HSF1) has been identified as the cellular component binding to these sequence elements. Under normal growth conditions, HSF1 exists as a phosphorylated monomer, in which DNA-binding and transcriptional activities are repressed. In response to heat shock and other chemical, environmental or physiological stresses, HSF1 undergoes trimerisation, binds to the HSE and exhibits transcriptional activity. Several studies have shown that the temperature at which HSF1 is activated is not fixed, which implies that additional factors play important roles in regulating the activity of this protein. The binding of HSF1 to HSE has been shown to be highly cooperative, deviations from the NGAAN consensus sequence are tolerated in vivo because multiple HSEs foster cooperative interactions between multiple HSF trimers. Sequence variations of the binding site affect the affinity of HSF1 for the HSE of a particular target gene, thereby fine-tuning the heat shock response. Thus direct comparison between a natural HSE from the human HSP70.1 promoter and an idealised sequence, revealed a 57 fold difference in binding affinity for HSF1.

Heat shock promoters have extensively been used in different experimental systems. The highly conserved nature of the heat stress response allows the use of heterologous promoters. Thus, Xenopus and mouse HSP70 promoters were first tested in the fish system, later followed by experiments with fish promoters. The main problem observed for these experiments revealed high levels of background activity for these promoters. On contrary to Drosophila, in vertebrates HSP70 promoters are highly expressed during certain stages of development, explaining the high basal level in these experiments. Generation of transgenic lines can alleviate this problem but transient injection experiments are hampered by the leakiness of the promoter.

Transient injection experiments constitute a fast gain-of-function method for fish and frog embryos. In fish embryos mRNA injection at the on-cell stage leads to uniform misexpression in the embryo, whereas injected DNA is subject to distribution phenomena, resulting in mosaic expression. Different modifications have therefore been tested to improve DNA distribution. The recently introduced meganuclease method results in elevated integration efficiency of the DNA into the genome (Ristoratore et al., Development 26, 3769-79). As a consequence, the integrated DNA is stably transmitted among the somatic cells, thereby largely increasing the level of misexpressing cells. Moreover, the number of transgenic offspring is drastically increased.

The WO 87/00861 relates to a heat shock control method whereby a recombinant DNA gene is functionally linked under the transcriptional and/or translational control of a heat shock control element. One control element is a heat shock promoter consensus region, whereby this region comprises not more than 11 deoxynucleotides of a formula C (T/G) (C/A) GnnnnTTC, whereby n is independently selected from A, T, C or G. Specific examples of mutant promoter regions are shown, whereby mutants SE 1-12 only contain synthetic consensus-like sequence elements in their promoters (AGAAGCTTCT) repeated 1 to 12 times. It was shown that a mutant SE7 containing 7 sequence elements is the most active in heat shocked cells.

In the WO 98/06864 also the control of gene expression using a heat shock protein promoter is described whereby it is mentioned that the heat shock element includes the sequence nGAAn, repeated at least 2 times in head-to-head or tail-to-tail orientation nGAAnnTTCn or nTTCnnGAAn.

The U.S. Pat. No. 5,614,399 relates to a method of inducibly enhancing the expression of a DNA sequence, whereby the DNA sequence is operably joined to a regulatory region comprised of a heat shock element and a promoter. The heat shock element is described as CTGGAATTTCTAGA. A heat shock regulatory region comprising multiple heat shock elements is disclosed.

The EP 0 159 884 B1 relates to a heat shock promoter comprising the consensus sequence CTXGAAXXTACXXX, whereby X is A, T, C or G.

The WO 87/04727 A1 relates to an inducible heat shock and amplification system, whereby the gene encoding for a polypeptide or protein is placed under the control of an inducible heat shock promoter. However, the heat shock promoter described in this document is isolated from a eukaryotic source and is therefore a natural and not artificially designed promoter.

The aim of the present invention is therefore to provide a promoter or regulatory element for protein expression which has superior properties to the known promoters and regulatory elements, in particular with low background activity, high inducibility and lack of tissue specific expression.

This aim is achieved with a DNA molecule which is characterised in that it comprises at least 2 consensus sequences, each consensus sequence consisting of 3 pentameric units, said pentameric units having a sequence XGAAY or an inverse sequence Y′TTCX′, X being selected from the group consisting of A, T, G, and C, and Y of at least one, preferably two, still preferred all three, of said 3 pentameric units of at least one consensus sequence being selected from the group consisting of A, T, and C, the Y of the remaining pentameric units of said at least one consensus sequence being selected from the group consisting of A, T, G, and C, whereby in the case that said DNA molecule comprises more than 6 consensus sequences, Y of all pentameric units is selected from the group consisting of A, T, G, and C. This DNA molecule has shown to be optimal in expression induction with low background activity, high inducibility and lack of tissue specific expression.

The term “DNA molecule” relates to a sequence which induces protein expression upon induction, whereby an additional, e.g. heterologous promoter may be present.

With respect to the inverse sequence “X′” relates to a nucleotide being complementary to the “X” of the non-inverse pentameric unit. This means that “X′” is selected from the group consisting of A, T, G and C. The “Y′” which is complementary to the “Y” of the non-inverse pentameric unit is therefore selected from the group consisting of T, A and G for at least one, preferably two, still preferred all, pentameric units of at least one consensus sequence, whereby the “Y′” of the remaining pentameric units is selected from the group consisting of A, T, G and C. Therefore, in the DNA molecule at least one pentameric unit, be it the inverse or non-inverse sequence, comprises either an Y being selected from A, T and C or an Y′ being selected from A, T and G. It has been shown that in the case that the DNA molecule comprises a lower number of consensus sequences, for example two to six consensus sequences, it is important that the consensus sequence shows optimal inducibility which is the case when Y is not a G or Y′ is not a C. However, in the case that the DNA molecule comprises a larger number of consensus sequences, e.g. more than six consensus sequences, the Y or Y′ may be selected from the group consisting of A, T, G and C, since the higher number of consensus sequences causes protein expression induction with superior properties. In other words: the lower the numbers of consensus sequences in the DNA molecule, the more it is important to provide an optimal pentameric unit which is the case, when Y is not G and Y′ is not C.

It is possible that one consensus sequence comprises only non-inverse pentameric units XGAAY or only inverse pentameric units Y′TTCX′. However, it is also possible that one consensus sequence comprises two non-inverse pentameric units and one inverse pentameric unit or one non-inverse pentameric unit and two inverse pentameric units. One consensus sequence may comprise identical pentameric units with respect to the X/X′ and Y/Y′. However, in one consensus sequence 2 or all 3 pentameric units may vary in the X/X′ and Y/Y′.

The DNA molecule may further comprise identical consensus sequences or non-identical consensus sequences or, in the case that there are three or more consensus sequences in the DNA molecule, two or more consensus sequences can be identical and the remaining consensus sequences different. The difference can be either with respect to the selection of the X and/or Y (Y′ and/or X′) or with respect to the presence of non-inverse and inverse sequences or both.

It is important that the DNA molecule comprises at least two consensus sequences. However, the DNA molecule may comprise more than 10, more than 20, more than 30, more than 40 or more than 50 consensus sequences. Furthermore, the DNA molecule may comprise additional sequences, sequence fragments or single nucleic acids which may be of any specific or non-specific sequence or even an additional pentameric unit. For example the DNA molecule may comprise 2 consensus sequences and an additional 1 or 2 pentameric units.

Preferably, the DNA molecule comprises 4-24, preferably 7-16, still preferred 8 consensus sequences. It was shown that these numbers of consensus sequences are optimal, since on the one hand the DNA molecule comprises a sufficient number of consensus sequences in order to show strong inducibility and on the other hand the DNA molecule is not too long to show negative side activities, like recombination and others.

Advantageously, the consensus sequences are separated by 2 to 10 bp, preferably by alternatingly 3 and 6 bp. It was found that the respective factor, e.g. heat shock factor, binds in an optimal manner, when the consensus sequences are not directly linked to one another. These short spacer sequences allow for specific binding and activation of the respective factor to each consensus sequence.

According to a preferred embodiment the middle pentameric unit of at least one, preferably each consensus sequence is an inverse sequence compared to the outer pentameric units, preferably sequence Y′TTCX′. This means that the middle pentameric unit may be the non-inverse or the inverse sequence, depending on whether the two outer sequences are inverse or non-inverse. By alternatingly providing a non-inverse and inverse sequence the respective factor binds strongly and shows high inducibility, whereby it is shown to be optimal when at least one, preferably each consensus sequence is as follows: XGAAY Y′TTCX′ XGAAY.

Advantageously, the X is C or G, still preferred A. In the case that X is a C or G, the respective factor shows excellent binding and activating properties, which are, however, even better in the case that X is an A. Accordingly, X′ is preferably G or C and still preferred T. This applies for at least one X of the whole DNA molecule, preferably several X of the DNA molecule, still preferred all X of the DNA molecule. A DNA molecule comprising pentameric units in which X is always A therefore shows ideal properties.

In a further advantageous DNA molecule Y is C. Accordingly, for the inverse sequence Y′ is preferably G. As mentioned above for X, this applies for at least one Y of the whole DNA molecule, preferably several Y of the DNA molecule, still preferred all Y of the DNA molecule. Therefore, a DNA molecule, in which all Y are a C shows optimal inducibility.

Advantageously, therefore at least 1, preferably all consensus sequences are AGAAC GTTCT AGAAC. As already mentioned above, in the case that the DNA molecule comprises 6 or less consensus sequences, it is preferable that all consensus sequences are as defined above. In the case that the DNA molecule shows more than 6 consensus sequences, it is possible that 1 or more pentameric units show the above mentioned variations of X or Y or the respective X′ or Y′, however, with similarly high performances.

A further aspect of the present invention relates to a regulatory molecule which comprises a DNA molecule according to the present invention as defined above and a promoter upstream and/or downstream of said DNA molecule. Since the DNA molecule is bidirectional the promoter can be placed on either side of the DNA molecule. Upon induction of the DNA molecule through binding with a respective factor the promoter(s) is (are) activated. Such a regulatory molecule is ideal for the use in specific inducible protein expression systems.

Still preferred said promoter is a minimal promoter, preferably CMV minimal promoter. The combination of the inventive DNA molecule together with a minimal promoter has shown to be optimal, in particular due to low background activity, and at the same time providing a highly inducible promoter.

A further aspect of the present application relates to a gene which comprises in its regulatory region the above inventive regulatory molecule. Upon induction of the promoter the protein polypeptide for which the gene codes is expressed. Hereby, the gene may be a sequence which codes for any protein or polypeptide. Said gene can code for example a protein, which is for example of therapeutical or analytical interest. However, it is also possible that said gene codes for a protein which is itself a regulatory element. Such a regulatory protein can be for example the Gal4-VP16 which is used in the amplification system as described by Köster and Fraser (Dev. Biol. 233, 329-346 (2001)), whereby the Gal4-VP16—once expressed—activates a promoter which expresses in an amplified manner any protein or polypeptide of interest. This definition of proteins which are expressed by the present system applies throughout the present application.

A further aspect of the present invention relates to a vector which comprises in its regulatory region the above inventive regulatory molecule. The vector is a polynucleic acid which comprises different DNA fragments and which is able to be propagated. Apart from the inventive regulatory molecule the vector preferably comprises a multiple cloning site into which any DNA sequence—in particular DNA sequences which code for proteins or polypeptides—may be inserted. Furthermore, vectors comprise defined restriction sites and preferably specific selection sequences, e.g. sequences which provide a resistance against an anti-biotic.

A further aspect of the present application relates to a construct which comprises an inventive regulatory molecule as defined above with two promoters, one promoter placed upstream and a second promoter placed downstream of said DNA molecule, one gene placed under the control of one promoter and a second gene placed under the control of said second promoter, said construct preferably comprising further globin UTRs and polyadenylation signals. This inventive construct will induce the DNA molecule—upon binding of a respective factor in particular heat shock factor—which will then activate both promoters after which both genes are expressed. Preferably, both promoters are identical in order to provide for identical activation of protein expression. Preferably, the two genes provided on the construct are different. For expression studies it is for example ideal to provide on the one hand a gene coding for luciferase which is used for sensitive quantification and on the other hand a gene which codes for Gfp which is used as an expression marker. These two genes have therefore complementary features so that the inventive construct can be designed to provide for an optimal system for expression studies.

A further aspect of the present invention relates to a cell, preferably a human, animal, plant, insect or yeast cell, which comprises an inventive gene, an inventive vector or an inventive construct as defined above. Hereby, the term “animal” relates also to cold-blooded animals, in particular fish and frogs. Of course, the cell may also be the cell of a microorganism, as for example a yeast cell. Due to the low background activity it is possible to carry out transient expression experiments due to the improved inducibility and reduced background activity which is not the case in conventional expression systems using for example the known heat shock promoter HSP70.

Preferably, said gene vector or construct is stably integrated in said cell. This can be for example carried out with the meganuclease method, since this results in elevated integration efficiency of the DNA into the genome. Therefore, the integrated DNA is stably transmitted among the somatic cells and the number of transgenic offspring is importantly increased.

A further aspect of the present application relates to a trans-genic plant, animal or insect, which comprises said stably transfected inventive gene, inventive vector or inventive construct as defined above. Under “plant, animal and insect” it is understood that these organisms can be at any stage of development, therefor, for example also larvae, seeds or embryos are comprised. Furthermore, also fragments of these organisms are comprised by this aspect, as for example leaves, roots, calli, eyes, etc.

A further aspect of the present invention relates to the use of a DNA molecule according to the present invention as defined above in an expression system, preferably inducible misexpression system, whereby an inventive gene, a vector or construct as defined above is inserted into a cell after which said cell is exposed to stress so that said promoter is activated to induce gene expression. Therefore it is possible to provide a system in which gene expression is inducible at any chosen moment since the DNA molecule is activated upon stress exposure after which the promoter is activated and induces gene expression. It was shown that the inventive DNA molecule in particular combined with a minimal promoter shows no or low background activity, high inducibility and lack of tissue specific expression which are required features for a misexpression system with superior properties. It is understood, that not only one single cell can be used but also a plurality of cells, which can be a cell culture, an organism, e.g. a plant, animal or insect or a fragment thereof. The term “insert” relates to any kind of method for integrating the gene, vector or construct into the cell or organism. This can be conventional transfection with particle gum or also an injection, e.g. micro-injection or other techniques. Furthermore, this use also relates to gene therapy, whereby the gene codes for a therapeutically active protein and is inserted into the organism to be treated. By exposure to stress, preferably after the gene is spread throughout the organism, whereby the stress can be applied locally, e.g. to a specific tissue or organ, the protein of interest is expressed at the specific area of the organism, e.g. a tumor tissue. Therefore, the inventive use is of particular interest for tumor therapy. In the case of an organism or tissue, the applied stress can also be high frequency irradiation which causes warming of the tissue and which is particularly gentle.

Preferably, said stress is heat, irradiation, dryness, elevated salt, organic compounds and heavy metal concentration, respectively. Of course, it is possible to combine 2 or more stresses as for example exposing the cell to heat and dryness or heat and elevated salt concentration, etc. Whether or not a stress is applied depends on the cell which is used. For example human cells are exposed to stress at a temperature which is higher than cold-blooded animal cells which show an increase in promoter activity already at a temperature of 35° C. The same counts for dryness, elevated salt and heavy metal concentration, since the optimal growth conditions of different cells vary considerably.

Still preferred, said insertion is a stable transfection. The person skilled in the art is able to design the optimal transfection protocol in order to achieve stable transfection, meaning that the specific sequence is stably integrated into the genome of the cells which leads to continuous expression in the progeny.

A further aspect of the present invention relates to a method for producing an expression system, preferably an inducible misexpression system, whereby an inventive gene, vector or construct as defined above is inserted into a cell, after which said cell is preferably cultured. Again, the term “insert” relates to any kind of method for integrating the gene, vector or construct into the cell. This can be conventional transfection or transformation methods depending on whether the cell is eukaryotic or prokaryotic, however the insertion can also be an injection, e.g. micro-injection or other techniques. Preferably, said cell is cultured after said insertion leading to a stably transfected cell line or, in case the cell was an embryo, into further developed stages of the respective organism, for example larvae and fish or similar. If the cell was stably transfected, the progeny will also comprise the inventive gene vector or construct.

Preferably said cell is a plant, animal, insect or human cell, whereby the same definitions and advantageous embodiments as above apply.

Still preferred, said cell is a fish or frog embryo and said culturing results are larvae and fish or frogs, respectively.

These systems have shown to be particularly advantageous for heat shock expression systems, since in mammals strict control of the body temperature makes the in vivo application of this system difficult. However, systems like fish or insects are ideal for the induction of a heat shock response at elevated temperatures.

Advantageously, said insertion is a stable insertion which results in a stable transgenic cell line. Here the same definitions and further embodiments as above apply.

Still preferred, said cell, preferably said cultured cell, is exposed to stress, said stress preferably being heat, dryness, elevated salt and heavy metal concentration, respectively. Also here the same preferred embodiments and definitions as above apply.

Advantageously, a meganuclease enzyme is co-inserted together with said gene, vector or construct into said cells. This results in an elevated integration efficiency of the DNA into the genome, so that the integrated DNA is stably transmitted among the somatic cells thereby largely increasing the level of misexpressing cells. Furthermore, the number of transgenic offspring is drastically increased.

Preferably, a method for gene therapy of an organism is provided whereby a gene, vector or construct according to the present invention is administered to said organism after which stress is, preferably locally, applied to said organism so that at least one protein is expressed in solid organism. Hereby, the administration of said gene, vector or construct can be carried out according to any known method for gene therapy, whereby it is preferable that said gene, vector or construct is spread throughout the organism, which is preferably a human being. Stress is preferably applied locally, so that the at least one protein of interest is expressed where it is therapeutically necessary. This gene therapy is of particular interest for the treatment of a tumor, since the protein of interest can be expressed specifically at and/or around the tumor. Of course, it is possible to express more than one protein. Furthermore, any kind of stress can be locally applied, in particular heat stress as well as irradiation, in order to warm the area of interest.

A further preferable aspect of the present application relates to a method for monitoring stress inducible substances whereby a gene, a vector or construct according to the present invention as mentioned above is inserted into a cell or cells after which the expression of said protein is detected. Here, as already defined above, a plurality of cells can be a cell culture or a whole organism, for example a plant, animal, insect or human being. One possibility is to detect whether or not the cell or cells is (are) exposed to stress inducible substances, in which case the expression of said protein is detected. Another possibility is to expose said cell (cells) to at least one stress inducible substance, after which the expression of said protein is checked. In case the expression of said protein is detected, this will indicate that the substance is stress inducible. Furthermore, this method can be used for the detection of location (in the cell or organism) the substance induces stress. Hereby the cell (cells) is (are) exposed to at least one stress inducible substance and after a certain amount of time of for example cell culture or breeding of the organism, the location of expression of said protein is detected. Said stress inducible substances are for example salts, organic compounds, (heavy) metals, etc.

The present invention is described in more detail with the help of the following examples and figures to which, however, it is not limited whereby

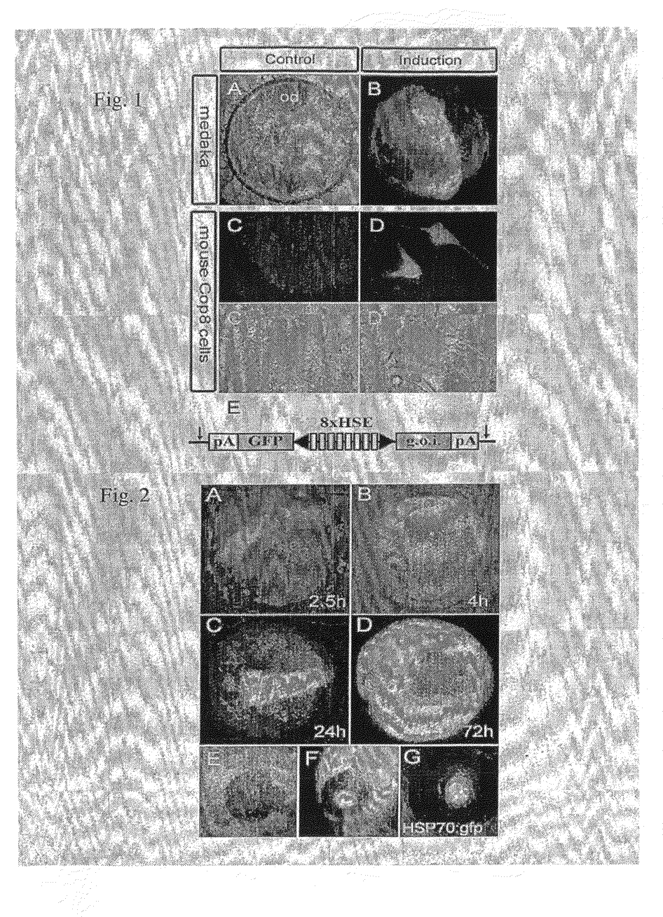

FIG. 1 shows the activation of the heat shock element (HSE) promoter in medaka embryos and in cell culture;

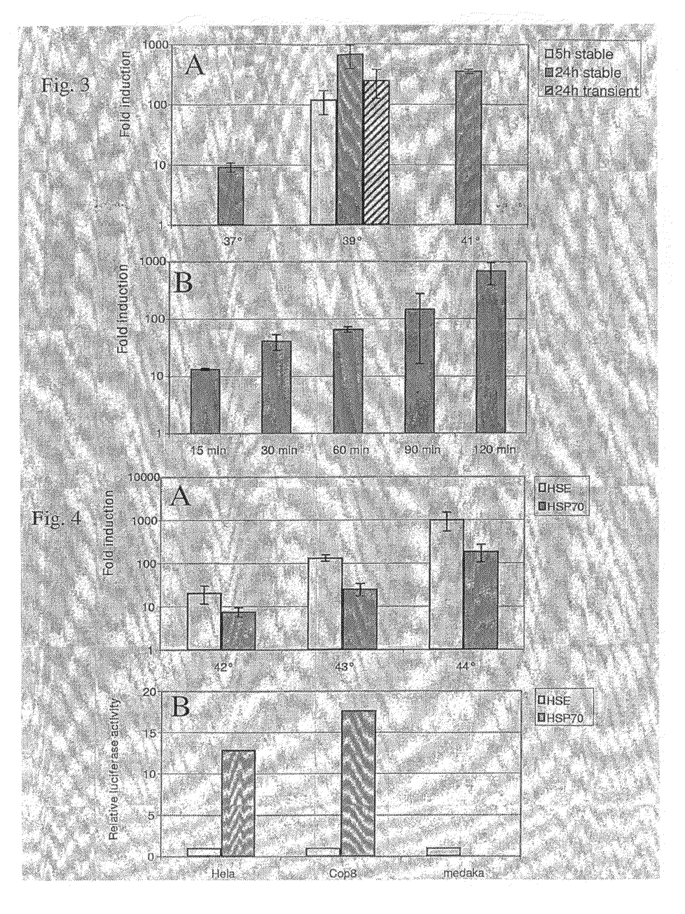

FIG. 2 shows a stable integration of a HSE construct into a medaka genome;

FIG. 3 shows the quantification of heat shock induction of the HSE promoter in vivo;

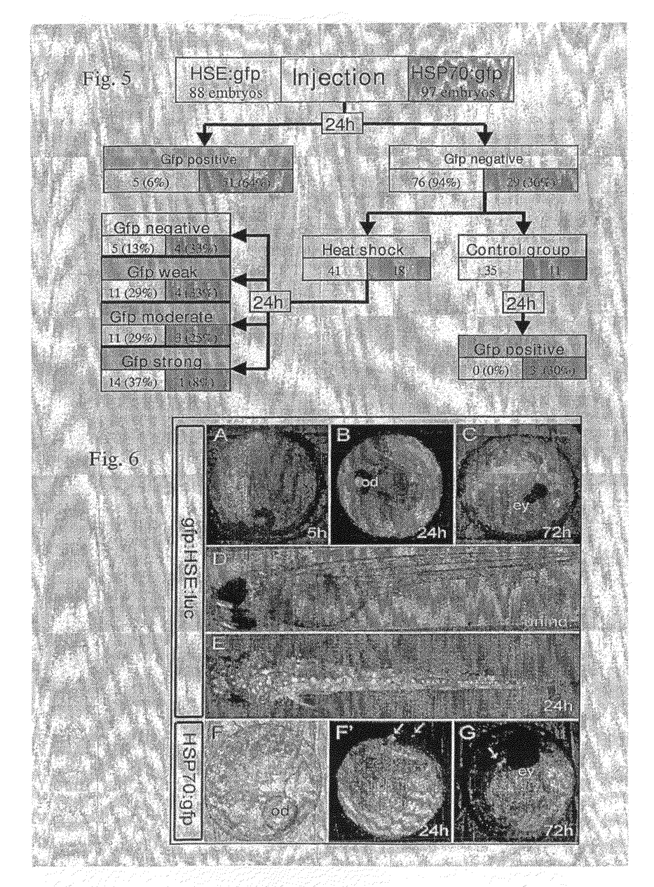

FIG. 4 shows the quantitative comparison of the HSE promoter with the Zebrafish HSP70 promoter;

FIG. 5 shows transient misexpression with heat stress inducible constructs;

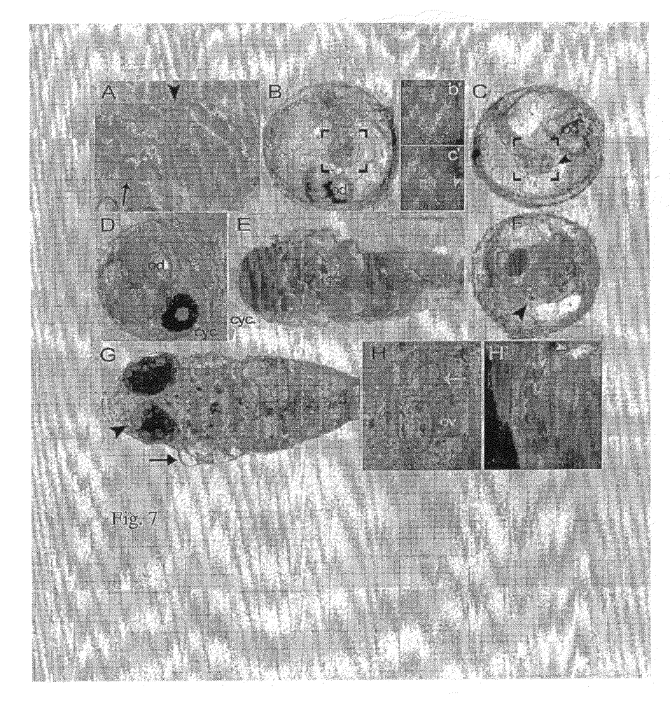

FIG. 6 shows a comparison between the HSE promoter and the HSP70 promoter in a typical transient experiment; and

FIG. 7 shows phenotypes of medaka embryos misexpressing Fgf8.

### EXAMPLES

### Example 1

### Production of Transgenic Cell Lines

Medaka embryos and adults of the Cab inbred strain were used for all experiments. Adult Fish were kept under a reproduction regime (14 hour light/10 hour dark) at 26° C. Embryos were collected daily immediately after spawning. Embryonic stages were determined according to Iwamatsu.

Multimerised heat shock elements (HSE) with the idealised sequence AGAACGTTCTAGAAC, alternatingly separated by 3 and 6 bp, were generated by oligonucleotide ligation. A fragment containing 8 HSEs was inserted upstream of a CMV minimal promoter, driving the firefly luciferase gene flanked by 5′ and 3′ globin UTRs and the SV40 polyadenylation (pA) signal. In the opposite orientation, a similar cassette containing gfp instead of the luciferase gene, but the same minimal promoter, UTRs and the pA signal was inserted, resulting in the gfp:HSE:luc construct. The gfp:HSE:Fgf8 construct was obtained by replacing the luciferase gene with the zebrafish Fgf8 cDNA. This cDNA with the same flanking sequences and pA signal was used to generate the CMV:Fgf8 construct using the complete CMV promoter/enhancer region of the pCS2 vector.

Fertilised medaka eggs were microinjected through the chorion into the cytoplasm at the one cell stage. After injection, the embryos were incubated at 28° C. mRNA was in vitro transcribed using the T7 message machine kit (Ambion) and injected in 1×Yamamoto buffer. DNA was prepared with a Jetstar midiprep-kit (Genomed) and also injected in 1× Yamamoto buffer. For the meganuclease system, DNA was co-injected with I-SceI meganuclease enzyme (0.5 unit/μl) in 1× I-SceI buffer (New England BioLabs). For all experiments, a pressure injector (FemtoJet, Eppendorf) was used with borosilicate glass capillaries (GC100-10; Clark Electromedical Instr.) pulled on-a Sutter Instruments P-97. Capillaries were backfilled with the injection solution.

To make transgenic lines, the gfp:HSE:luc construct was injected at 10 ng/μl together with I-Scel meganuclease (0.5 units/μl) into embryos at the one-cell stage. For screening 60 larvae of 14 days were heat treated at 39° C. for 1 h and observed under the fluorescent microscope after 1 day. 17 larvae were gfp-positive and the 8 with the strongest expression were selected. After 8 weeks the mature fish were crossed with wild-type fish and their F1 progeny was assayed for transgene expression after heat shock. 4 of the 8 selected fish produced progeny that exhibited gfp fluorescence following heat induction. The average germlinetransmission rate was different between each founder (10-27%). The founder with the highest germline transmission rate (27%) was selected for analysis of the F1 offspring.

Human Hela and mouse Cop8 cells were kept under standard cell culture conditions with DMEM medium supplemented with 10% FCS. 1×10⁵ cells were transfected in a 24 well plate with 400 ng DNA (if necessary filled up with pBS plasmid) and 0.5 μl Transfast (Promega) in 200 μl medium without FCS. As an internal reference, 5 ng of a Renilla luciferase expression vector (SV40:Rluc) were co-transfected. After 6 hours the medium was replaced by fresh DMEM+FCS. Heat treatment was applied after 24 hours by transferring the plates in a different incubator (without CO2). The cells were lysed 24 hours after the heat shock and luciferase activity measured with the Dual Luciferase Kit (Promega). For normalisation, firefly luciferase activity values were sub-sequently divided by Renilla luciferase values.

### Example 2

### Heat-Shock Treatment and Luciferase Activity Measurement of Medaka Embryos

For heat-treatment, 10-20 embryos or 5 larvae were incubated in 0.5 ml of Embryo Rearing Medium (ERM) in a 1.5 ml tube at the elevated temperature in a heating block. After this treatment, the embryos were transferred into petri dishes and kept at 28° C. For luciferase activity measurements (usually 24 hours after the heat shock), the embryos were transferred individually into 1.5 ml tubes, homogenised with a pestle in 100 μl of lysis buffer, incubated for 15 min on a shaker at RT and then centrifuged for 5 min at 14000 rpm (RT). Luciferase activity was determined from the supernatant with the Dual Luciferase Kit (Promega).

Heat shock at 37° C. for 2 hours resulted in few gfp-positive cells after 24 hours, therefore higher temperatures were tested. Indeed, after treatment at 39° C. for 2 hours substantial gfp expression could be observed in the embryos (FIG. 1B), whereas a control group did not show ectopic gene activation (FIG. 1A; C=control, I=induction; m=mouse). Luciferase activity measurements for these embryos furthermore demonstrated bidirectional promoter activity of the construct. Similarly, an experiment in mouse Cop8 cells confirmed heat stress inducibility of the present construct in a different system (FIGS. 1C and 1D): The gfp:HSE:luc DNA construct was co-injected with meganuclease into one-cell stage medaka embryos (A, B) or transfected in mouse Cop8 cells (C, C′, D, D′). The injected embryos were divided into a control group and a test group. Embryos of the untreated control group (A) were gfp negative. Embryos of the test group were treated at 39° C. for 2 h resulting in strong gfp expression (B). Typical embryos (stage 24) are shown for both groups. After transfection into mouse Cop8 cells, control plates remained gfp negative (C). Treatment at 44° C. for 2 h induced a strong gfp response in the transfected cells (D). C′ and D′ are the corresponding brightfield views for C and D, respectively. A, B, C and D are fluorescent images, background light was added for image A to visualise the otherwise gfp negative embryo. A schematic presentation of the HSE promoter is depicted in (E). The artificial promoter contains 8 multimerised heat shock elements flanked by two minimal promoters in opposed orientation. Gfp on one side and the gene of interest (luciferase or Fgf8) at the other side are expressed from the bicistronic promoter. The vector is flanked by I-Scel meganuclease sites (arrows). Abbreviations: od, oil droplet; pA, SV40 polyadenylation signal; HSE, heat shock element; g.o.i., gene of interest.

### Example 3

### Generation of a BSE Transgenic Medaka Line

In order to thoroughly analyse the properties of the HSE promoter in vivo, the gfp:HSE:luc construct was stably integrated into the medaka genome. All transgenic embryos of 4 independent transgenic medaka lines were completely devoid of basal gfp expression at all stages of development, but developed strong gfp fluorescence in the whole embryo after heat shock treatment (FIGS. 2A-2D). Quantitation revealed similar expression levels and induction rates for all 4 lines, thereby excluding position effects of transgene integration. All transgenic embryos developed normally. One transgenic line was selected for further experiments. 2.5 hours after treatment of the embryos at 39° C., gfp was first detectable under the fluorescent microscope (FIG. 2A). The signal intensity increased up to 24 h and due to the stability of the protein persisted for several days (FIGS. 2B-2D). Induced expression was seen in all embryonic tissues, including the lens (FIG. 2F), whereas lenses of uninduced embryos lacked any gfp activity (FIG. 2E). Basal gfp expression in the lens is typically observed for HSP70:gfp transgenic zebrafish in the uninduced state and can be explained by a combined effect of high promoter activity and low protein turnover in this tissue. Indeed, injection of a zebrafish HSP70:gfp construct confirmed the preferential activation of the uninduced promoter in the medaka lens (FIG. 2G). Therefore, the HSE promoter can be efficiently induced in all embryonic tissues, without showing any background activity.

### Example 4

### Properties of the HSE promoter and Comparison with the Zebrafish HSP70 Promoter

Making use of the high reproducibility of the transgenic line, various conditions for activation of the HSE promoter were tested in a quantitative manner. For this purpose the luciferase gene of the bicistronic promoter construct was used. Transgenic embryos were collected and incubated at 28° C. 24 hours past fertilisation, when the embryos finished gastrulation (stage 19), heat treatment was initiated. 24 h later the embryos were lysed and luciferase activity was measured. Even for this highly sensitive marker, activity measurements of uninduced control embryos were close to the detection limit, confirming the low background activity of the HSE promoter. In a first series of experiments, the temperature of heat treatment was varied. Luciferase activity measurements revealed a 9.3 fold increase in promoter activity after treatment at 37° C. for 2 hours, compared to untreated control embryos kept at 28° C. (FIG. 3A; Fi=Fold induction; s=stable; t=transient). The strongest response (up to 680 fold induction) was obtained at 39° C. Decreasing the time between heat shock treatment and lysis of the embryos, from 24 to 5 hours, resulted in a concomitant 5.5 fold reduction of luciferase activity (at 39° C., FIG. 3A). Luciferase activity was measured 5 hours (5 h stable) or 24 hours after heat treatment (24 h stable). For comparison, a transient injection experiment with the same construct was quantified identically (24 h transient). The induction is displayed in a logarithmic scale. Duration of the heat treatment at 39° C. was varied in (B), luciferase activity was measured 24 hours after induction. For calculation of the values (transgenic embryos) between 2 and 7 independent measurements were taken (5 for the uninduced control used as reference) and 17 for the transient medaka experiment (20 for the uninduced control). No further increase in induction rates was observed for 41° C., whereas 42° C. treatment resulted in extensive death of embryos (data not shown). The same survival rates were obtained for uninjected control embryos, indicating that 2 hours at 41° C. is the limit for heat treatment of medaka embryos. Using the optimal temperature of 39° C. the duration of the heat shock treatment was then varied. A gradual increase of the induction rate was observed starting from 15 minutes (13.5 fold) up to 2 h of treatment (680 fold, FIG. 3B). Therefore, the HSE promoter is highly inducible when stably integrated into the medaka genome, with an optimal activation temperature at 39° C. Luciferase activity measurements of embryos injected at the one cell stage with gfp:HSE:luc DNA revealed an average 250 fold induction upon a 2 hour 39° C. treatment. Taking into account the variabilities of injection experiments, this result is in good agreement with the data for the transgenic line (FIG. 3A).

In mammalian cells which show optimised growth rates at 37° C., induction of the heat shock response has been described for 42° C., but elevated activities have been observed for temperatures up to 44° C. When this temperature was applied for 2 hours to mouse Cop8 cells, a 22 fold activation of luciferase activity for cells transiently transfected with the HSE construct (FIG. 4A) was observed. This induction rate is weak compared to the values obtained for medaka embryos. Therefore the temperature of the heat shock treatment was increased and indeed a 134 fold induction at 43° C. was observed. At 44° C. the response was even more pronounced (1020 fold induction, FIG. 4A), but in some experiments partial cell death was observed at this temperature. This toxic effect might be attributed to the high level of gfp expression in these cells, since untransfected control cells survived this treatment. Other types of cellular stress like heavy metals similarly lead to strong activation of the construct (100 μM Cd⁺⁺). These data demonstrate a high inducibility of the HSE construct also in cell culture cells. In order to compare these data to a natural heat shock promoter, a construct containing a 1.5 kb fragment of the zebrafish HSP70 promoter driving the luciferase gene was used. In cell culture experiments this construct showed high inducibility upon heat treatment. Nevertheless, in all experiments the absolute numbers of HSP70 promoter induction were clearly below that for the HSE construct under comparable conditions (FIG. 4A; Fi=Fold induction; Rla=Relative luciferase activity). The idealised sequence and the multimerisation of the HSE thus increased the inducibility on average 5 fold compared to the natural promoter. Similarly, an improved induction rate for the construct in injection experiments into medaka embryos compared to the HSP70 construct was observed.

Beside improving the inducibility, a rationale of the present approach was to reduce the background activity of the promoter. To test this, luciferase activity values of the uninduced control cells transfected was compared with both constructs. Due to the complex structure of the HSP70 promoter and known tissue specific expression characteristics, different cell lines were tested and in addition quantified medaka injection experiments. In all cases, the HSP70 promoter showed dramatically higher background activity compared to the artificial HSE construct (FIG. 4B). The observed differences were between 13 and 18 fold for cell culture cells (Hela and Cop8, respectively) and 12 fold for in vivo injection experiments. Taken together, the artificial HSE construct shows improved inducibility together with reduced background expression.

### Example 5

### A Transient Misexpression System for Medaka Embryos Based on the HSE Promoter

The heat shock promoter has proven to be a valuable tool for inducible misexpression in fish embryos. Nevertheless, stable integration into the genome is necessary to overcome the problem of high background activity of this promoter. Compared to simple DNA injection experiments, the generation of transgenic lines is time consuming. Reduced background activity and high inducibility make the HSE promoter an ideal candidate for an application in transient experiments. An additional tool applied for these experiments was a recently developed method based on the restriction enzyme meganuclease, which leads to more uniform expression after injection of DNA.

The DNA construct gfp:HSE:luc was injected into one-cell stage medaka embryos. After 24 hours, when they had passed gastrulation (stage 19), the embryos were scored for background expression under the fluorescent microscope. In a typical experiment, 6% of the embryos showed weak gfp activity (FIG. 6). This background activity was restricted to less than 10 cells and depended on the injection conditions. Background up to 24% was seen for experiments where the embryos were partially injured by non-optimal injection needles, whereas values down to 0% were obtained for the best experiments. Furthermore, the number of gfp-positive cells decreased with time, suggesting that the majority of these cells underwent cell death. The positive embryos were excluded from further analysis and the remaining gfp-negative embryos were divided into two groups. One group served as an uninduced control group, whereas the other group was heat treated at 39° C. for 2 hours. None of the embryos of the control group developed any gfp signals during further development. On contrary, 87% of the heat treated embryos were positive after 24 h (FIG. 6) and more than one third of these embryos showed strong gfp activity (FIGS. 5A-5C; e=embryo; Inj.=Injection; n=negative; p=positive; w=weak; mod=moderate; str.=strong; Hs=Heat Shock; C=control group). Gfp expression was recorded 5 hours (A), 24 hours (B) and 72 hours (C) after induction. Uninduced embryos were grown until hatching and did not show any gfp expression (D). Yellow staining originates from autofluorescing cells. These larvae were induced (39° C./1 h) and exhibited a strong response after 24 hours (E). For comparison, embryos were injected the same way with the HSP70:gfp construct and induced under the same conditions (F, F′, G). Gfp signals were preferentially observed in yolk cells (F′), gfp positive cells within the embryo are marked by arrows. (G) The same embryo 48 hours later. F is a brightfield view of F′. Abbreviations: od, oil droplet; ey, eye. The groups of strong, moderate and weak gfp activity mainly differed by the number of positive cells, but not the intensity of expression within individual cells. Spot-wise misexpression in individual cells or cell clones as observed in the weak gfp group is furthermore an important aspect of the technique for certain experimental questions. In all cases, misexpression was mainly confined to the embryo (FIGS. 5A-5C), whereas strong gfp signals in yolk cells were rarely observed. Taken together, in this typical injection experiment (88 embryos injected), a group of 36 embryos exhibited induced misexpression, out of which 14 showed widespread activation and a control group of 35 uninduced embryos was devoid of any misexpression (FIG. 6).

In order to directly compare these results, a similar experiment with the zebrafish HSP70 promoter was performed. The same DNA backbone including the gfp gene, flanking UTRs, polyadenylation signal and meganuclease sites, was used for the construct. Upon injection, 64% of the medaka embryos showed background gfp activity after 24 hours (FIG. 6). In the majority of cases, widespread expression occurred in yolk cells indicating a preference of the HSP70 promoter for this tissue. Excluding the gfp-positive embryos, the remaining embryos were again divided into 2 groups. 30% of the uninduced control group developed a gfp signal within 24 hours, confirming the high background activity of this promoter. In the heat treated group, 66% of the embryos were positive after 24 hours. In all cases preferential activity was observed for yolk cells, making the detection of positive cells in the embryo difficult (FIGS. 5F-5G). In absolute numbers, only 1 out of 97 embryos injected with the HSP70 construct showed strong induced misexpression (FIG. 6). This has to be compared to 14 embryos of this group for the HSE promoter experiment. Therefore, due to the high background activity of this promoter, both the evaluation of the uninduced control group is difficult and the number of strongly expressing embryos within one experiment is largely reduced. A transient application of the natural HSP70 promoter is therefore of limited use.

DNA injection typically leads to a mosaic distribution of the expression constructs. The high percentage of embryos with wide-spread activation of the HSE transgene has to be attributed both to the high inducibility of the promoter and the meganuclease method. Elevated integration rates for the injected DNA constructs into the genome of the early embryo are responsible for the latter effect. Whereas this results in a gradual shift to more widespread misexpression during early development, a more dramatic difference is observed at later stages. For conventional injection techniques, misexpression is almost lost within a few days of development. Due to stable integration into the genome of somatic cells, the meganuclease system can lead to continuous misexpression in larvae and adult fish. It was tested, whether the combination of the meganuclease system with the HSE promoter can be used to obtain inducible misexpression at late stages of development. 60 embryos were injected with the gfp:HSE:luc DNA construct at the one-cell stage and then grown until stage 40 (14 days), where they all were gfp-negative (FIG. 5D). After heat treatment at 39° C. for 1 hour 28% of these larvae exhibited moderate or strong gfp expression (FIG. 5E). Therefore the HSE promoter can be used in combination with the meganuclease system to study late developmental processes by induced misexpression in transient experiments.

### Example 6

### Misexpression of Fgf8 with the Transient HSE System

For a first application of the present inducible misexpression system, the Fgf8 gene was selected. A gfp:HSE:Fgf8 construct containing the zebrafish Fgf8 CDNA together with the gfp marker gene bidirectionally expressed from the same promoter was injected into one-cell stage embryos at different concentrations together with meganuclease. At a concentration of 5 ng/μl 45% of the heat treated embryos were gfp positive, whereas no embryo of the uninduced control group showed gfp expression (Table 1). The marker gene expression equalled the developmental defects caused by Fgf8 misexpression. All surviving embryos of the control group appeared normal, whereas 23% of the heat treated animals developed morphological defects (Table 1). Increasing the DNA concentration to 12 ng/μl and 25 ng/μl had little effect on gfp expression, but the higher Fgf8 dose directly influenced the frequency of affected embryos (up to 43%). Similarly, the elevated amounts of DNA resulted in the appearance of malformed embryos in the control group (up to 10%). Therefore, the extent of developmental effects induced by the HSE system can be influenced by DNA dosage. For highly effective genes like Fgf8, a low concentration is necessary to start induction in embryos where the level of misexpression is below the detection limit, as concluded from the absence of any morphological defects in the control group. On the other hand, higher concentrations can be helpful to detect more dramatic phenotypes due to the high level of misexpression.

TABLE 1
Dose dependence of HSE induced misexpression of Fgf8 in medaka
embryos

| Concentation | Heat shock | Gfp | Developmental | Normal | Dead | Number of |
|---|---|---|---|---|---|---|
| gfp:HSE:Fgf8 | 39° C./2 h | expression | Defects | Embryos | Embryos | Embryos |
| 5 ng/μl | + | 45% | 23% | 62% | 14% | 109 |
| | − | 0% | 0% | 92% | 7% | 67 |
| 12 ng/μl | + | 56% | 56% | 35% | 36% | 60 |
| | − | 1% | 1% | 98% | 0% | 58 |
| 25 ng/μl | + | 47% | 43% | 26% | 30% | 96 |
| | − | 4% | 10% | 60% | 10% | 46 |

The spectrum of observed malformations for Fgf8 misexpression was in good agreement with published roles for Fgf8 in different tissues. At a low frequency the formation of a secondary axis was observed, abnormalities of the pectoral and the tail fin and problems with the blood circulatory system and the heart. Phenotypes affecting the eyes and the otic vesicles appeared more often and were therefore analysed in more detail. Inducible misexpression systems offer the advantage to investigate gene function during different time windows, which differ by the responsiveness of individual tissues to various levels of the ectopic gene activity. In the next series of experiments the time of induction (Table 2) was systematically varied. In addition, injection of Fgf8 mRNA and a CMV:Fgf8 construct was included into these experiments. Thus, ectopic gene activation starting from the one cell stage (mRNA), mid-blastula stage (CMV:Fgf8) and various time points during and shortly after gastrulation with the HSE induction system was covered. The resulting eye phenotypes are summarised in Table 2.

TABLE 2
Eye phenotypes observed after Fgf8 misexpression

| HSE:Fgf8 (12) | 2 somites (19) | 3 (12) | 1 | 0 | 0 | 0 | 25 |
|---|---|---|---|---|---|---|---|
| HSE:Fgf8 (5) | Mid-gastrula (15) | 13 (19) | 1 | n.d. | n.d. | 1 | 67 |
| HSE:Fgf8 (12) | Pre-mid-gastrula (14) | 17 (48) | 9 | 0 | 2 | 5 | 35 |
| HSE:Fgf8 (5) | Pre-mid-gastrula (14) | 17 (24) | 13 | 6 | 4 | 2 | 70 |
| HSE:Fgf8 (25) | Early gastrula (13) | 23 (50) | 7 | 0 | 4 | 2 | 46 |
| CMV:Fgf8 (5) | Mid-blastula (10) | 29 (39) | 20 | 14 | 1 | 0 | 74 |
| CMV:Fgf8 (25) | mid-blastula (10) | 4 (4) | 2 | 1 | 1 | 0 | 91 |
| Fgf8 mRNA (25) | One-cell | 23 (45) | 17 | 14 | 0 | 0 | 51 |
| | | Development | Eye | | Pigmentation | Cyclopic | Injected |
| Construct (ng/μl) | Stage of activation¹ | Al defects² (%) | Defects³ | Loss of eye | Defect In eye | Eye | Embryos |
| Fgf8 mRNA (5) | One-cell | 15 (37) | 4 | 2 | 0 | 0 | 40 |

¹Embryonic determined according to lwamatsu (1994) are written in brackets
²Totla number of embryos with visible developmental defects in percent
³Total number of embryos with eye defects
n.d., not detected

The typical phenotype observed after injection of Fgf8 mRNA was a complete loss of eyes often accompanied by a dysgenesis of the forebrain (Table 2, Fgf8 mRNA 25 ng). This dramatic phenotype was seen at a similar frequency upon injection of Fgf8 DNA expression constructs (CMV:Fgf8 25 ng), indicating that this tissue is competent to respond to Fgf8 until the mid-blastula stage. On the contrary, activation of the protein at a slightly later stage with the HSE promoter (early gastrulation, Table 2), does not result in this phenotype any more. Whereas, various effects on eye size were observed for all stages of activation (FIG. 7F), the complete loss of both eyes was associated mainly with early misexpression. Injection of Fgf8 mRNA and CMV:Fgf8 results predominantley in a strong eye/forebrain phenotype (A). In most cases the forebrain is dramatically reduced, but more posterior structures appear normal (the position of the mid-hindbrain boundary is marked by an arrowhead and an otic vesicle by an arrow). (B and C) show examples of observed eye phenotypes after induction of the gfp:HSE:Fgf8 DNA at stage 14. (b′ and c′) gfp expression in the area encompassing the black square marks in the corresponding images (B and C). Embryos developing cyclopic eyes (B) exhibit misexpression not within the eye, but in the adjacent tissue (b′). Loss of one eye (C) correlated with misexpression on the same side of the embryo (c′), the other eye is marked by arrowheads. (D, E) show the embryo with the cyclopia phenotype at a later embryonic stage (D) and a larval stage (E). An embryo induced at the 2-somite stage (stage 19) exhibited reduced size of one eye marked by an arrowhead (F). A pigmentation phenotype in one eye (arrowhead) and an expanded otic vesicle (arrow) was seen for a larva induced during mid-gastrula (G). Ectopic otic vesicles were repeatedly observed, a magnification of such an embryo is shown in (H); the ectopic otic vesicle is marked by an arrow. Note, that gfp, marking the Fgf8 misexpressing cells, is not seen within in the vesicle (H′); autofluorescing medaka cells are marked by an arrowhead. Abbreviations: cyc., cyclopic eye; od, oil droplet. Interestingly different eye phenotypes appeared when Fgf8 expression was induced slightly later. Pigmentation defects in the eye (FIG. 7G) and the formation of cyclopic eyes (FIGS. 7A, 7B, 7D and 7E) accumulated for induction times between early and mid gastrulation (Table 2). Both phenotypes were not observed for mRNA injections. This does not depend on the high dose of protein obtained after mRNA injection, since a reduction of the amount of injected mRNA leads to the same phenotypes, but at a reduced frequency (Table 2, Fgf8 mRNA 5 ng).

A major advantage of the HSE construct is that misexpressing cells can be traced by their gfp signal. Thus gfp activity was observed in cells directly adjacent to the cyclopic eyes (FIGS. 7A and 7B), but interestingly, no gfp signal occurred within these eyes. In other experiments, the pattern of gfp appeared more restricted, consequently confining dramatic phenotypes to these parts of the embryo. In FIG. 7C an example is shown, where misexpression was found in the right half of the embryo resulting in loss of the eye specifically at this side. This transient approach therefore represents a straightforward approach to follow misexpressing cells or cell clones and study developmental consequences caused by positional effects. Based on loss-of-function experiments, Fgf signalling could be associated with multiple steps in ear formation of zebrafish embryos, which starts during somitogenesis with induction of the otic placode and later the otic vesicle. In the zebrafish, Fgf8 is not expressed in the placode prior to the 18 somite stage, but experiments based on mRNA injection and Fgf8-bead implantation, provided evidence that Fgf8 acts as a placode inducer acting from the hindbrain primordium. However, induction of ectopic otic vesicles through overexpression of Fgf8 alone was not possible with these methods and also failed in chick embryos. Applying the HSE system, frequently both expanded and duplicated otic vesicles after activation of Fgf8 expression in the midgastrula stage (FIGS. 7G and 7H) were observed. Consistent with the idea that fgf8 acts as an inducing agent from the distance, gfp-positive cells appeared not within, but adjacent to the otic vesicles (FIG. 7H′). Other phenotypes, like the reduction of the otolith number, were seen preferentially for mRNA injected embryos.

Fgf8 has multiple roles during various stages of embryonic development. Induced misexpression of Fgf8 with the HSE system is a valuable tool to study these functions. Here Fgf8 misexpression mainly served to study the basic requirements for a transient inducible system. Many interesting questions concerning Fgf8 gene function might be investigated with this tool.

### SUMMARY OF RESULTS

The artificial HSE promoter was highly active, both after heat treatment and exposure to heavy metal ions, indicating that the full response to cellular stress can indeed be mediated by isolated HSEs. Therefore, on contrary to previous studies, it could be shown that HSEs are sufficient to mediate a full stress response and that other elements of heat shock promoters contribute predominately to basal expression, but not the inducibility. Furthermore, multimerization and optimization of the HSEs leads to improved inducibility, compared to natural promoters. The combination with a TATA box results in a minimal size inducible promoter, which can be used in a bidirectional manner.

Superior Properties of the HSE Promoter upon Heat Shock Treatment

Three parameters are of major importance for the application of an inducible promoter: 1) low background activity, 2) high inducibility and 3) lack of tissue-specific expression. In the uninduced state, the activity of the promoter has to be as low as possible in order to prevent any unspecific effects. Tissue-specific preferences of the promoter can further complicate the situation by increasing the background in certain tissues. Upon induction, the promoter should provide a sufficiently high activity, resulting in ubiquitous expression. Low background activity and high expression are quite contradictory properties for a promoter. Quantitation of these two extreme levels and calculation of the inducibility is therefore a good measurement for the applicability of the promoter. The HSE promoter was tested in transient experiments and in stable transgenic lines, in cell culture cells as well as in medaka embryos. Luciferase activity measurements were used to allow sensitive quantitation and gfp expression to follow expression patterns during development. In all these assays the HSE promoter demonstrated superior properties.

Natural heat shock promoters like the HSP70 promoter have successfully been used for induced misexpression during embryonic development. Leakiness in the uninduced state is the main disadvantage of this promoter and results in high background activity. By reducing the complex structure of heat shock promoters, it was possible to dramatically diminish the background expression. A high basal level can be attributed to promoter elements like CCAAT- and SP1 boxes, which are known to provide ubiquitous expression (CCAAT Xenopus papers). It was therefore expected that the absence of these elements should result in a reduced background level in all cells. Comparison of basal luciferase activity values for both promoters in various cell lines and in vivo, indeed, showed a more than 10 fold average reduction in background activity for the artificial HSE construct.

The direct comparison between the HSE and the HSP70 promoter should provide clear data on the applicability of our construct. Beside a reduced background, the artificial HSE promoter exhibited improved inducibility in all experiments. On average, a 5 fold increase for this important parameter was observed. Up to 1000 fold activation leads to high levels of misexpression even under less favourable conditions including DNA injection, which impose a higher variability to the experiments.

Endogenous heat shock promoters are developmentally regulated. The tissue-specific components of these promoters have not been characterized in detail, but result for example in preferential expression of the HSP70 promoter in the yolk and the lens. High transcriptional activity in the yolk was in these experiments the predominant problem for a transient application of the HSP70 promoter in medaka embryos. Removal of all non-inducible sequences successfully eliminated all tissue-specific components from the HSE promoter, which therefore exhibited equal expression levels throughout the whole embryo. Background expression in the lens could also be eliminated, an important factor for misexpression experiments in the developing eye. These results clearly demonstrate, that the elevated background expression in yolk and lens cells does not depend on a high basal level of cellular stress response acting on the HSEs, but depends on other sequence elements in the HSP70 promoter.

Summarising the improvements which were obtained for the artificial HSE promoter compared to the natural version, it was possible to reduce the general background activity, increase the inducibility and eliminate all tissue specific components.

### Optimizing the Conditions of Heat Treatment

As expected increased temperatures and longer exposure to the stress factor results in an elevated response. In order to obtain high expression levels, excessive cellular stress has to be applied, which can be harmful to the cells and in extreme cases lead to cell death. In particular mammalian cells seem to tolerate deviations from their optimal growth conditions less well. Activation of the heat stress response was weak (20 fold) at 42° C. At a slightly higher temperature (44° C.) this value dramatically increased to 1000 fold activation. This seems to be the limit, since increasing numbers of cell death were observed upon extended exposure to this temperature. Fish embryos tolerate different temperatures more easily. Normally kept at 26-28° C., a first heat stress response is seen in medaka embryos at 37° C. (10 fold). Again, raising the temperature by only 2 degrees, the induction level jumped to the maximum value of 680 fold. Even upon extended incubation at 41° C. the embryos developed normally and finally at 42° C., the embryos died. Therefore, in vivo application of the heat shock response in medaka embryos is a straightforward approach. Treatment at 39° C. leads to optimal induction rates, retaining a reasonable distance to 42° C., where the embryos die.

Comparing these results with the literature, quite similar data have been obtained for zebrafish embryos. For heterologous HSP70 promoters, peak induction values were obtained at 39° C. and using the endogenous promoter a temperature of 40° C. was found to be optimal. The HSP70 promoter has also been tried in a combination with the Gal4-UAS system. The amplification effect of Gal4-VP16 drastically reduces the duration of the heat treatment, allowing short pulses of activation, but does not eliminate the background problem of this promoter, in particular during transient applications.

### A Transient Misexpression System Based on the HSE Promoter

Transient misexpression experiments are a fast way to study gene function in vivo. Injection of mRNA, which is translated immediately, often results in dramatic early phenotypes. Application of DNA constructs shifts the initiation of expression to the mid-blastula stage, but in order to study gene function at later developmental stages, an induction system has to be used. Problems with reproducibility of the injection procedure and distribution phenomena affecting the DNA copy number, make the transient application of induction systems difficult. Only systems of superior quality can compensate for these problems. Heat shock promoters represent an attractive single component induction system. When the HSP70 promoter was tested in transient experiments, high background expression was observed. On the contrary, the artificial HSE promoter shows improved inducibility and a largely reduced background activity and can therefore efficiently be used for transient experiments in fish embryos.

In a typical transient experiment close to 100 embryos were injected. Less than 10 embryos show weak background activation of gfp and are eliminated. The remaining embryos are divided into a control group and a test group. At the required developmental stage, embryos of the test group are heat treated and obtain high levels of misexpression within a few hours after induction. About 40 misexpressing embryos can be expected. Due to application of the meganuclease method, a high proportion of these embryos shows widespread activation of the transgene. At the same time, the control group can be analysed. Due to the improved inducibility of the HSE promoter, low amounts of DNA can be injected, which avoids the appearance of any phenotypes before induction. The level of expression can be regulated by the duration of the heat treatment.

A particular advantage of the HSE construct is the coexpression of the gfp marker gene from the same promoter. Bicistronic expression requires a short and symmetric structure of the DNA molecules, whereas most natural promoters have a strong tendency for unidirectional transcription. Thus, addition of a TATA box to the 5′ end of the actin promoter resulted in uneval expression levels. A strong activation of the HSE promoter was observed in both orientations. Coexpression of gfp is an important tool to eliminate embryos with background expression and allows the immediate recognition of promoter activation in the test and the control group. Furthermore, the exact position of misexpressing cells in the embryo can be determined. This experimental design therefore provides an efficient tool for gene function analysis in fish embryos

Misexpression of Fgf8 with the Transient HSE Misexpression System

Early misexpression of Fgf8 in medaka resulted in a dramatic phenotype. The embryos did not develop eyes and a severe dysgenesis of the forebrain was observed. Surprisingly, mRNA injection experiments in the zebrafish exhibited a quite different phenotype. The embryos showed abnormalities along the dorsoventral axis. Even in the most severe cases, where posterior structures of the embryo became lost, anterior structures like the eyes and the forebrain remained intact. Since we used the zebrafish cDNA for our medaka experiments, Fgf8 protein function can not account for these differences. Medaka embryos seem to have a divergent competence of the forebrain tissue to react to elevated levels of Fgf8. Interestingly, similar eye/forebrain phenotypes were observed for En2 misexpression experiments in medaka. Overexpression of En2 in these embryos activates in the forebrain a genetic programme comparable to that acting in the mid-hind-brain boundary. Both En2 as well as Fgf8 are part of this genetic cascade and might therefore both be able to activate the same pathway. A similar phenotype was observed for En misexpression in Xenopus, whereas mRNA injections of Eng2 into zebrafish embryos resulted in a mild phenotype, not activating any mid-hind-brain boundary specific genes in the forebrain. Therefore, both medaka and Xenopus differ from zebrafish by the competence to activate the mid-hindbrain boundary genetic programme in an ectopic position. Normally, this latent pathway is not activated during embryonic development, but misexpression of En and possibly also Fgf8 can trigger the genetic cascade, leading to dramatic consequences. Zebrafish lack this competence, which might be due to the absence of a component essential for the pathway. This has no further consequences on development, since this pathway is normally not activated in the forebrain. This might explain, why misexpression of both En or Fgf8 has no dramatic consequences on forebrain development in zebrafish.

Fgf8 is an example of a highly active gene with multiple functions during embryonic development. Early overexpression with mRNA or DNA injection leads to severe phenotypes, which block further analysis of later functions. Application of a transient inducible system solves this problem. Delayed activation of Fgf8 with the HSE system thus prevented the severe early phenotype (complete loss of the eyes) observed after mRNA/DNA injection experiments and allowed the study of late Fgf8 functions, in particular in the eye. Thus, the formation of cyclopic eyes, which has not yet been described for Fgf8 misexpression experiments, was observed. Interestingly, in medaka, a similar pheno-type was observed for overexpression of a dominant negative Fgf receptor, interfering with Fgf signalling. Therefore, both activating as well as blocking Fgf8 function leads to the same phenotype. A similar observation was recently made for Fgf8 dependent cell survival in the mouse forebrain. In these experiments, both, reduction of gene dosage, as well as overexpression resulted in the same phenotype (apoptotic cell death).

The otic placode is induced by signals from the neighbouring hindbrain during early somitogenesis. Fgf signalling molecules have been implicated in this process and recent studies suggest that Fgf3 and Fgf8 act in a redundant fashion during the ear induction. Combined inactivation of the two genes in zebrafish by using the acerebellar (Fgf8) mutant, morpholino knock-down, or by inhibition of Fgf-Signalling with SU5402 treatment completely blocks ear development. Gain-of-function experiments further strengthened the role of Fgf family members in this inductive event. Ectopic otic vesicle formation was observed in overexpression experiments for Fgf2 and Fgf3 in Xenopus, for Fgf3 in chick embryos and Fgf10 in the mouse. Surprisingly, similar attempts for Fgf8 by mRNA injection and Fgf8-bead implantation failed, both in fish and chick embryos. Applying the HSE system it was possible to induce additional otic vesicles in medaka. Fgf8 misexpression was induced in these embryos during mid-gastrulation, which is in good agreement with previous studies, timing the inductive event to this stage. In addition to the exact timing, the expression level and the position of the signal can be of critical importance for successful induction. Indeed, tracing of gfp activity as a marker for misexpressing cells confirmed the action of Fgf8 from a distance in these experiments. On contrary to duplication, frequently malformations of the otic vesicle were observed, which appeared most prominently in mRNA experiments. mRNA injection typically leads to uniform misexpression, suggesting that broad overexpression of Fgf8 including the developing ear might result in this phenotype. Implantation of beads better resembles an inductive event from the distance, but it is difficult to test all possible positions and protein levels. Transient DNA injection experiments, on the other hand, provide a large spectrum of variations both in the expression level and the position of misexpressing cells. Having in addition the option to manipulate the timing of activation, the HSE system is ideal for such experiments. Applying this technique, hundreds of embryos, each with slightly different parameters for misexpression can rapidly be scanned within a few experiments. Furthermore, the position and intensity of the gfp signal can be traced in vivo.

Summarising both the data on quantitation of luciferase and gfp activity, together with the results for inducible misexpression of Fgf8 during embryonic development, the HSE promoter perfectly matches the requirements for a transient inducible system. Such a system is able to study gene function during later stages of development, in particular when early overexpression results in dramatic phenotypes. Time windows of competence to react to a signal can rapidly be investigated. Applying this promoter in transient injection experiments in combination with the meganuclease system furthermore extends the spectrum of expression patterns from spot-wise misexpression in single cells, preferentially seen for inductive events from a distance, up to widespread overexpression during all stages of development. Expression level and position of the misexpressing cells can readily be followed in vivo.

1-26. (canceled)

27. A DNA molecule comprising at least 2 consensus sequences, each consensus sequence consisting of 3 pentameric units, each of the pentameric units comprising a sequence XGAAY or an inverse sequence Y′TTCX′, wherein:
each X is A, T, G, or C, and
Y of at least one of the pentameric units of at least one consensus sequence is A, T, or C, and Y of the remaining pentameric units of the at least one consensus sequence is A, T, G, or C;
wherein, if the DNA molecule comprises more than 6 consensus sequences, Y of all pentameric units is A, T, G, or C.

28. The DNA molecule of claim 27, further defined as comprising 4 to 24 consensus sequences.

29. The DNA molecule of claim 28, further defined as comprising 7 to 16 consensus sequences.

30. The DNA molecule of claim 29, further defined as comprising 8 consensus sequences.

31. The DNA molecule of claim 27, wherein the consensus sequences are separated by 2 to 10 bp.

32. The DNA molecule of claim 31, wherein the consensus sequences are separated by alternatingly 3 and 6 bp.

33. The DNA molecule of claim 27, wherein the middle pentameric unit of at least one consensus sequence is an inverse sequence compared to the outer pentameric units.

34. The DNA molecule of claim 33, wherein the middle pentameric unit is of sequence Y′TTCX′.

35. The DNA molecule of claim 33, wherein the middle pentameric unit of all consensus sequences are an inverse sequence compared to the outer pentameric units.

36. The DNA molecule of claim 27, wherein at least one X is C or G.

37. The DNA molecule of claim 27, wherein at least one X is A.

38. The DNA molecule of claim 27, wherein Y is C.

39. The DNA molecule of claim 27, wherein at least one consensus sequence is AGAAC GTTCT AGAAC.

40. The DNA molecule of claim 39, wherein all of the consensus sequences are AGAAC GTTCT AGAAC.

41. The DNA molecule of claim 27, further defined as comprised in a regulatory molecule comprising a promoter upstream and/or downstream of the DNA molecule.

42. The DNA molecule of claim 41, wherein the promoter is a minimal promoter.

43. The DNA molecule of claim 41, wherein the promoter is a CMV minimal promoter.

44. The DNA molecule of claim 27, further defined as comprised in a regulatory region of a gene.

45. The DNA molecule of claim 27, further defined as comprised in a vector.

46. The DNA molecule of claim 27, further defined as comprised in a construct having one promoter placed upstream and a second promoter placed downstream of the DNA molecule, one gene placed under the control of one promoter and a second gene placed under the control of the second promoter.

47. The DNA molecule of claim 46, wherein the construct further comprises at least one globin UTR and/or polyadenylation signal.

48. The DNA molecule of claim 27, further defined as comprised in a cell.

49. The DNA molecule of claim 48, wherein the cell is a human, non-human animal, plant, insect or yeast cell.

50. The DNA molecule of claim 48, wherein the DNA molecule is further defined as comprised in a gene, a vector, or a construct in the cell.

51. The DNA molecule of claim 50, wherein the gene, vector or construct is stably integrated in the cell.

52. A transgenic plant, animal, or insect, comprising the DNA molecule of claim 27.

53. A method of producing an expression system, comprising introducing a nucleic acid comprising a DNA sequence of claim 27 into a cell.

54. The method of claim 53, further comprising culturing the cell.

55. The method of claim 53, wherein the expression system is further defined as an inducible misexpression system.

56. The method according to claim 53, wherein the cell is a plant, animal, insect or human cell.

57. The method of claim 53, wherein the cell is a fish or frog embryo, and the culturing results in larvae and fish or frogs, respectively.

58. The method of claim 53, wherein the introducing results is a stable transgenic cell line.

59. The method of claim 53, further comprising stressing the cell.

60. The method of claim 59, wherein stressing the cell comprises exposing it to heat, dryness, elevated salt concentration and/or heavy metal concentration.

61. The method of claim 53, further comprising co-inserting a meganuclease enzyme into the cell.

62. A method of gene therapy comprising administering a nucleic acid comprising a DNA segment of claim 27 and encoding a protein to an organism and stressing the organism, wherein the protein is expressed in the organism.

63. The method of claim 62, wherein stressing the organism is further defined as administering local stress to the organism.

64. A method of monitoring stress inducible substances comprising inserting a nucleic acid comprising a DNA segment of claim 27 and encoding a protein into at least one cell and detecting expression, if any, of the protein in the cell.
